(19) United States
(12) Patent Application Publication
(10) Pub. No.: US 2017/0052162 A1
(43) Pub. Date: Feb. 23, 2017

(54) DEVICES AND METHODS FOR DETECTING HALOGENATED ORGANIC COMPOUNDS

(71) Applicant: The Regents of the University of California, Oakland, CA (US)

(72) Inventors: Hye-Won Shin, Irvine, CA (US); Marc Madou, Irvine, CA (US); Dan M. Cooper, Irvine, CA (US); Donald R. Blake, Irvine, CA (US)

(73) Assignee: The Regents of the University of California, Oakland, CA (US)

(21) Appl. No.: 15/242,355

(22) Filed: Aug. 19, 2016

Related U.S. Application Data
(60) Provisional application No. 62/208,439, filed on Aug. 21, 2015.

(51) Int. Cl. G01N 33/00; G01N 27/407

(52) U.S. Cl. CPC G01N 33/0049; G01N 27/4074; G01N 27/4075; G01N 27/4071

(57) ABSTRACT

Disclosed herein are devices comprising a solid state gas sensor comprising a catalyst and a detection component capable of sensing halogenated gases through an increase or decrease in electrical conductivity. Further disclosed herein are methods of detecting one or more halogenated organic compounds, comprising providing a device comprising a solid state gas sensor comprising a catalyst and a detection component capable of sensing halogenated gases through an increase or decrease in electrical conductivity, and using the device to detect one or more small solid state halogenated organic compounds. Also disclosed herein are various methods of treating asthma in an individual.

### CROSS REFERENCE TO RELATED APPLICATIONS

This present application claims the benefit of priority from U.S. provisional application Ser. No. 62/208,439, filed Aug. 21, 2015, which is incorporated by reference in its entirety.

### FIELD OF THE INVENTION

The present disclosure is in the medical field, specifically as it relates to asthma inhalers.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Asthma inhalers, such as inhaled corticosteroids and bronchodilators, are the first line of asthma treatment. However, one problem facing clinicians and researchers has been the lack of technologies to measure treatment compliance and/or efficacy. Currently the typical methods to measure ICS or bronchodilator compliance include patient self-report of medication use and medication canister weighing or counting actuations. However, these current solutions are not optimal for both adults and children. Patient self-report of asthma inhaler use is notoriously inaccurate. Other advanced methods such as electronic monitoring of inhaled medication compliance are limited because it will not be able to detect whether the medication is actually inhaled. There is no readily accessible method available to identify asthma inhaler compliance or treatment efficacy. Thus, there is a need in the art for novel and effective methods and devices for identifying asthma inhaler compliance and treatment efficacy.

### SUMMARY OF THE DISCLOSURE

Various embodiments include a device, comprising a gas sensor comprising a catalyst and a detection component capable of detecting one or more halogenated gases by electrical conductivity. In one embodiment, the gas sensor is a solid state gas sensor. In one embodiment, the detection component is a sintered metal oxide. In one embodiment, the sintered metal oxide is SnO₂, TiO₂, or ZnO. In one embodiment, the detection component is a solid electrolyte. In one embodiment, the solid electrolyte is LaF₃. In one embodiment, the solid electrolyte selectively detects halogenated gases through a change in potentiometric voltage when fluorine adsorbs onto the solid electrolyte sensor's surface by rapidly exchanging fluoride ions. In one embodiment, the solid state gas sensor measures the level of HFA-134a in a sample. In one embodiment, the device is a handheld device. In one embodiment, the solid state gas sensor is a semiconductor oxide gas sensor and/or Taguchi sensor. In one embodiment, selectivity is achieved based on nature of the catalyst used. In one embodiment, selectivity is achieved based on the choice of semiconductor oxide and/or solid electrolyte. In one embodiment, selectivity is achieved based on the temperature of operation. In one embodiment, the device identifies asthma inhaler compliance and/or treatment efficacy. In one embodiment, the detection component is TiO₂ and/or ZnO. In one embodiment, the device further comprises a readout component that indicates the amount of HFA-134a in a sample.

Various embodiments also include a method of detecting one or more halogenated organic compounds, comprising providing a device comprising a gas sensor comprising a catalyst and a detection component capable of detecting one or more halogenated gases by measuring electrical conductivity; and using the device to detect one or more halogenated organic compounds. In one embodiment, the one or more small solid state halogenated organic compounds comprises hydrofluoroalkane (HFA). In one embodiment, the device is handheld. In one embodiment, measuring electrical conductivity comprises detecting an increase or decrease of electrical conductivity when gases are adsorbed on the sensor's surface. In one embodiment, the detection component comprises a solid electrolyte that selectively detects halogenated gases through a change in potentiometric voltage when fluorine adsorbs onto the solid electrolyte sensor's surface by rapidly exchanging fluoride ions.

Various embodiments further include a method of treating asthma in an individual, comprising: providing a sample of air from the individual's breath; detecting one or more halogenated organic compounds from the sample by using a device comprising a gas sensor comprising a catalyst and a detection component capable of detecting halogenated gases by electrical conductivity; and treating for asthma in the individual if one or more halogenated organic compounds are detected. In one embodiment, the electrical conductivity is proportional to the amount of halogenated gas present in the individual's breath sample. In one embodiment, the amount of halogenated gas in the individual's breath assists the individual in complying with an asthma medication regimen. In one embodiment, the one or more small solid state halogenated organic compounds comprise hydrofluoroalkane (HFA). In one embodiment, the device is handheld.

In one embodiment, provided herein is a method of determining asthma inhaler compliance, comprising providing a sample of air from the individual's breath, detecting one or more halogenated organic compounds from the sample by using a device comprising a gas sensor comprising a catalyst and a detection component capable of detecting halogenated gases by electrical conductivity; and determining asthma inhaler compliance based on detection of one or more halogenated organic compounds. In one embodiment, the gas sensor is a solid state gas sensor. In one embodiment, the solid state gas sensor comprises a solid electrolyte that selectively detects halogenated gases through a change in potentiometric voltage when fluorine adsorbs onto the solid electrolyte sensor's surface by rapidly exchanging fluoride ions. In one embodiment, the electrical conductivity is proportional to the amount of halogenated gas present in the individual's breath sample. In one embodiment, the one or more small solid state halogenated organic compounds comprises hydrofluoroalkane (HFA).

### DESCRIPTION OF THE DRAWINGS

Exemplary embodiments are illustrated in referenced figures. It is intended that the embodiments and FIGURE disclosed herein are to be considered illustrative rather than restrictive.

FIG. 1 illustrates, in accordance with embodiments herein, a schematic of a sensor system.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As used herein, the term “HFA-134a” refers to a hydrofluoroalkane, an aerosol propellant commonly present in inhalers.

As readily apparent to one of skill in the art, the technology is not limited to asthma inhalers, and can extend to any other area where a small solid state halogenated organic compound detector is the more appropriate solution for checking leaks of those compounds.

As disclosed herein, the inventors have developed various devices, including in one embodiment, an inexpensive hand held device that can measure a wide range of HFA-134a levels in human breath. In one embodiment, the present invention provides a device comprising a gas sensor composed of a catalyst and a sintered metal oxide and/or a solid electrolyte that detects one or more halogenated gases by electrical conductivity. In one embodiment, the gases are adsorbed on the sensor's surface. In some embodiments, the gas sensor is a solid state gas sensor. In another embodiment, the sintered metal oxide is SnO₂, TiO₂, and/or ZnO. In one embodiment, solid electrolyte is LaF₃. In one embodiment, the solid electrolyte selectively detects halogenated gases through a change in potentiometric voltage when fluorine adsorbs onto the solid electrolyte sensor's surface by rapidly exchanging fluoride ions. In one embodiment, LaF₃ is used when the organic compounds to be detected contain fluorine (F) atoms. In one embodiment, the solid state gas sensor measures a range of HFA-134a levels. In one embodiment, the device is a handheld device. In one embodiment, the solid state gas sensor is a semiconductor oxide gas sensor and/or Taguchi sensor. In one embodiment, selectivity is achieved based on nature of the catalyst used, choice of semiconductor oxide or solid electrolyte, and temperature of operation. In one embodiment, the device identifies asthma inhaler compliance and/or treatment efficacy. In one embodiment, the detection component is TiO₂ and/or ZnO. In one embodiment, the device further comprises a readout component that indicates the amount of HFA-134a in a sample. In another embodiment, the HFA gas sensor response is optimized by altering temperature and/or catalyst. In another embodiment, the solid electrolyte embodiment is more specific, works at lower temperatures and has a lower limit of detection (LOD) compared to a device without a solid electrolyte embodiment.

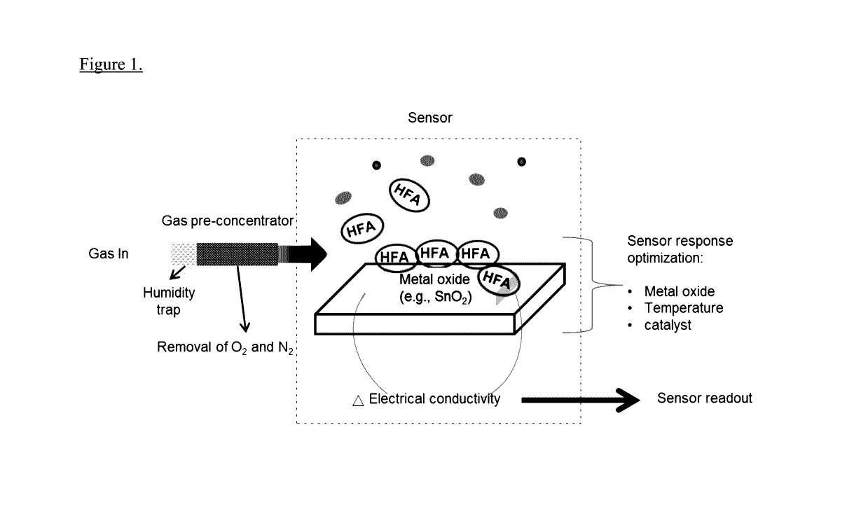

In another embodiment, the present invention provides a sensor system described in FIG. 1 herein.

In one embodiment, the present invention provides a method of detecting one or more halogenated organic compounds, comprising (a) providing a device comprising a gas sensor comprising a catalyst and a detection component capable of sensing halogenated gases by measuring electrical conductivity; and (b) using the device to detect one or more halogenated organic compounds. In one embodiment, the gas sensor is solid state. In one embodiment, the one or more small solid state halogenated organic compounds comprise hydrofluoroalkane (HFA). In another embodiment, the device is handheld. In one embodiment, measuring electrical conductivity comprises detecting an increase or decrease of electrical conductivity when gases are adsorbed on the sensor's surface. In one embodiment, the detection component comprises a solid electrolyte that selectively detects halogenated gases through a change in potentiometric voltage when fluorine adsorbs onto the solid electrolyte sensor's surface by rapidly exchanging fluoride ions.

In one embodiment, provided herein is a method of treating asthma in an individual, comprising: providing a sample of air from the individual's breath; detecting one or more halogenated organic compounds from the sample by using a device comprising a gas sensor comprising a catalyst and a detection component capable of detecting halogenated gases by electrical conductivity; and treating for asthma in the individual if one or more halogenated organic compounds are detected. In one embodiment, the electrical conductivity is proportional to the amount of halogenated gas present in the individual's breath sample. In one embodiment, the amount of halogenated gas in the individual's breath assists the individual in complying with an asthma medication regimen. In one embodiment, the one or more small solid state halogenated organic compounds comprise hydrofluoroalkane (HFA). In one embodiment, the device is handheld.

In one embodiment, provided herein is a method of determining asthma inhaler compliance, comprising (a) providing a sample of air from the individual's breath, (b) detecting one or more halogenated organic compounds from the sample by using a device comprising a gas sensor comprising a catalyst and a detection component capable of detecting halogenated gases by electrical conductivity; and (c) determining asthma inhaler compliance based on detection of one or more halogenated organic compounds. In one embodiment, the gas sensor is a solid state gas sensor. In one embodiment, the solid state gas sensor comprises a solid electrolyte that selectively detects halogenated gases through a change in potentiometric voltage when fluorine adsorbs onto the solid electrolyte sensor's surface by rapidly exchanging fluoride ions. In one embodiment, the electrical conductivity is proportional to the amount of halogenated gas present in the individual's breath sample. In one embodiment, the one or more small solid state halogenated organic compounds comprises hydrofluoroalkane (HFA).

The present invention is also directed to a kit for detection of one or more halogenated organic compounds. The kit is useful for practicing the inventive method of measuring asthma inhaler compliance, for example. The kit is an assemblage of materials or components, including at least one of the inventive compositions.

The exact nature of the components configured in the inventive kit depends on its intended purpose. For example, some embodiments are configured for the purpose of treating asthma. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as detecting one or more small solid state halogenated organic compounds, or treatment of asthma, for example.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing components used in conjunction with the detection of HFA, for example. The packaging material generally has an external label which indicates the contents and/or purpose of the kit and/or its components.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Hand Held Devices

In accordance with various embodiments herein, the invention is a hand held device that can measure a wide range of HFA-134a (the inhaler's aerosol propellant) levels in human breath. The technology developed can also impact other areas where a small solid state halogenated organic compound detector is the more appropriate solution for checking leaks of those compounds. The central component in the handheld device is a solid state gas sensor composed of a catalyst and a sintered metal oxide (e.g. SnO₂) or a solid electrolyte (e.g., LaF₃ in the case that the organic compounds contain F atoms) that detects halogenated gases through an increase or decrease in electrical conductivity when gases are adsorbed on the sensor's surface. The HFA gas sensor response is optimized by altering temperature and/or catalyst. The solid electrolyte version of the invention is more specific, works at lower temperatures and has a lower limit of detection (LOD).

### Example 2

### Sensors

In accordance with various embodiments herein, the central component in this handheld device is a semiconductor oxide gas sensor or a solid electrolyte. In one embodiment, the handheld device is the size of a breath alcohol sensor. In one embodiment, a Taguchi sensor may be used as the semiconductor oxide gas sensor. In one embodiment, the solid electrolyte is LaF₃. LaF₃ is particularly useful in the detection of organic compounds containing fluorine (F) atoms. In one embodiment, the solid electrolyte selectively detects halogenated gases through a change in potentiometric voltage when fluorine adsorbs onto the solid electrolyte sensor's surface by rapidly exchanging fluoride ions. In one embodiment, when the sensor of the device is heated to about 150° C., in the case of a solid electrolyte, or about 180° C., in the case of a semiconductor oxide, the device detects gases through an increase or decrease in electrical conductivity when gases of interest are adsorbed on the sensor's surface. Selectivity was achieved by three means: nature of the catalyst, nature of the detection component (such as, semiconductor oxide or solid electrolyte type), and temperature of operation. In terms of selectivity the solid electrolyte type is not only more selective it is also more sensitive and can operate at lower temperatures but is more difficult to fabricate and commands a higher purchase price. Both types can be further improved by absorbing the most hydrophilic gas species such as water and alcohols out of the breath. Commercially available Taguchi sensors were tested for optimal temperature range and specificity to HFA gases and were housed in a hand-held device also equipped with a moisture trap and an optical carbon dioxide sensor. In another embodiment, Taguchi sensors were further optimized, such as, by catalyst addition to the sintered metal oxide semiconductor, by changing the sensor temperature operating regime, or by using a small array of 2 to 4 Taguchi sensor elements. In one embodiment, the solid state gas sensor comprises a solid electrolyte that selectively detects halogenated gases through a change in potentiometric voltage when fluorine adsorbs onto the solid electrolyte sensor's surface by rapidly exchanging fluoride ions. The target metal oxides in this mode of operation included but were not limited to tin dioxide (SiO₂), titanium dioxide (TiO₂), zinc oxide (ZnO). The device was operated with the right catalyst and in the right temperature range, typically above 180° C. To ascertain their correct operation, solid-state gas sensing responses were compared to GC-MS methodology to measure breath HFA concentration from humans. Carbon dioxide concentration was measured as a reference breath gas. The humidity trap improved the Taguchi gas sensor selectivity. In one embodiment, the semiconductor oxide was replaced by a solid electrolyte making the sensor more selective and more sensitive at lower temperature of operation.

### Example 3

### Some Advantages

In one embodiment, an important aspect of this invention is to use hydrofluoroalkane (HFA) in the exhaled human breath as a potential biomarker of asthma inhaler compliance. Breath HFA is attractive because:

1) Breath HFA can be an objective measure of inhaler compliance because HFA is the most commonly used volatile aerosol propellant (CFC alternative since 1996) in metered dose inhalers to effectively deliver asthma medication to the lung

2) One can measure HFA concentration in the exhaled breath for at least 48 hours after a typical single inhalation, a useful interval in the clinical setting.

3) HFA is biologically inactive aerosol propellant, and is mainly eliminated by exhalation.

4) It is non-invasive and allows for either real time detection or assessment later at a remote location.

5) If the sample has been filtered during collection, it poses, unlike blood, little if any biohazard to healthcare workers or laboratory technicians

In one embodiment, the present invention presents unique precision of breath HFA levels as low as parts-per-trillion (pptv) to as high as parts-per-million (ppm), far exceeding previously reported HFA levels found either by the manufacturers or in the literature.

In another embodiment, the handheld sensor device is used in a clinic, ED, or at home. In one embodiment, the device presented herein can be used in a manner similar to the use of a breath alcohol sensor or a fire alarm. This can transform care of adults and children with asthma.

In another embodiment, the present invention impacts other areas where a small solid state halogenated organic compound detector is the more appropriate solution for checking leaks of those compounds.

### Example 4

### Schematic of a Sensor System

In accordance with various embodiments herein, one embodiment of the device for measuring the level of HFA in a gas is described in FIG. 1. The first component of the device comprises a humidity trap that removes some or all of the moisture from the gas. The gas then passes through a pre-concentrator which removes some or all of the O₂ and N₂. Following that, the gas enters the central component—a sensor system comprising a catalyst and a detection component. In one embodiment, the detection component is a metal oxide such as SnO₂, TiO₂, or ZnO. In another embodiment, the detection component is a solid electrolyte such as LaF₃. The HFA in the gas adsorbs on the sensor's surface, and reacts with it in the presence of the catalyst, resulting in a change in electrical conductivity compared to a gas without HFA. The change in electrical conductivity can be related to measuring the level of HFA in the gas—a large change in the electrical conductivity is indicative of a higher level of HFA in the gas. Finally, sensor readout is provided in the device indicating the level of HFA in the gas. The sensor response is optimized by optimizing the metal oxide used, the reaction temperature, and the catalyst used.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A device, comprising:
a gas sensor comprising a catalyst and a detection component capable of detecting one or more halogenated gases by electrical conductivity.

2. The device of claim 1, wherein the gas sensor is a solid state gas sensor.

3. The device of claim 1, wherein the detection component is a sintered metal oxide.

4. The device of claim 3, wherein the sintered metal oxide is SnO₂, TiO₂, or ZnO.

5. The device of claim 1, wherein the detection component is a solid electrolyte.

6. The device of claim 5, wherein the solid electrolyte is LaF₃.

7. The device of claim 5, wherein the solid electrolyte selectively detects halogenated gases through a change in potentiometric voltage when fluorine adsorbs onto the solid electrolyte sensor's surface by rapidly exchanging fluoride ions.

8. The device of claim 1, wherein the solid state gas sensor measures the level of HFA-134a in a sample.

9. The device of claim 1, wherein the device is a handheld device.

10. The device of claim 1, wherein the solid state gas sensor is a semiconductor oxide gas sensor and/or Taguchi sensor.

11. The device of claim 1, wherein selectivity is achieved based on nature of the catalyst used.

12. The device of claim 1, wherein selectivity is achieved based on the choice of semiconductor oxide and/or solid electrolyte.

13. The device of claim 1, wherein selectivity is achieved based the temperature of operation.

14. The device of claim 1, wherein the device identifies asthma inhaler compliance and/or treatment efficacy.

15. The device of claim 1, wherein the detection component is TiO₂ and/or ZnO.

16. The device of claim 1, further comprising a readout component that indicates the amount of HFA-134a in a sample.

17. A method of detecting one or more halogenated organic compounds, comprising:
providing a device comprising a gas sensor comprising a catalyst and a detection component capable of detecting one or more halogenated gases by measuring electrical conductivity; and,
using the device to detect one or more halogenated organic compounds.

18. The method of claim 17, wherein the one or more small solid state halogenated organic compounds comprises hydrofluoroalkane (HFA).

19. The method of claim 17, wherein the device is handheld.

20. The method of claim 17, wherein measuring electrical conductivity comprises detecting an increase or decrease of electrical conductivity when gases are adsorbed on the sensor's surface.

21. The method of claim 17, wherein the detection component comprises a solid electrolyte that selectively detects halogenated gases through a change in potentiometric voltage when fluorine adsorbs onto the solid electrolyte sensor's surface by rapidly exchanging fluoride ions.

22. A method of treating asthma in an individual, comprising:
providing a sample of air from the individual's breath;
detecting one or more halogenated organic compounds from the sample by using a device comprising a gas sensor comprising a catalyst and a detection component capable of detecting halogenated gases by electrical conductivity; and
treating for asthma in the individual if one or more halogenated organic compounds are detected.

23. The method of claim 22, wherein the electrical conductivity is proportional to the amount of halogenated gas present in the individual's breath sample.

24. The method of claim 22, wherein the amount of halogenated gas in the individual's breath assists the individual in complying with an overall asthma medication regimen.

25. The method of claim 22, wherein the one or more small solid state halogenated organic compounds comprises hydrofluoroalkane (HFA).

26. The method of claim 22, wherein the device is handheld.

27. A method of determining asthma inhaler compliance, comprising:
providing a sample of air from the individual's breath;
detecting one or more halogenated organic compounds from the sample by using a device comprising a gas sensor comprising a catalyst and a detection component capable of detecting halogenated gases by electrical conductivity; and
determining asthma inhaler compliance based on detection of one or more halogenated organic compounds.

28. The method of claim 27, wherein the gas sensor is a solid state gas sensor.

29. The method of claim 28, wherein the solid state gas sensor comprises a solid electrolyte that selectively detects halogenated gases through a change in potentiometric voltage when fluorine adsorbs onto the solid electrolyte sensor's surface by rapidly exchanging fluoride ions.

30. The method of claim 27, wherein the electrical conductivity is proportional to the amount of halogenated gas present in the individual's breath sample.

31. The method of claim 27, wherein the one or more small solid state halogenated organic compounds comprises hydrofluoroalkane (HFA).
